SEQUENCE LISTING (17 sequences)

SEQ ID NO: 1 - DNA, 22 bp, synthetic construct
  source 1..22: mol_type = other DNA
gcagtgcctatccagaaagtcc

SEQ ID NO: 2 - DNA, 23 bp, synthetic construct
  source 1..23: mol_type = other DNA
ggaatgaagtccaagccagtgac

SEQ ID NO: 3 - DNA, 22 bp, synthetic construct
  source 1..22: mol_type = other DNA
gaagccaccagctaaactgcag

SEQ ID NO: 4 - DNA, 23 bp, synthetic construct
  source 1..23: mol_type = other DNA
ctgacagcttgatgccaacatcg

SEQ ID NO: 5 - DNA, 22 bp, synthetic construct
  source 1..22: mol_type = other DNA
agaccgattggtgactctggag

SEQ ID NO: 6 - DNA, 23 bp, synthetic construct
  source 1..23: mol_type = other DNA
cctcggtgaataactgttgctgg

SEQ ID NO: 7 - DNA, 22 bp, synthetic construct
  source 1..22: mol_type = other DNA
gccactacctcaacctggtcac

SEQ ID NO: 8 - DNA, 22 bp, synthetic construct
  source 1..22: mol_type = other DNA
tcgctgtcgtctgtgaagagca

SEQ ID NO: 9 - DNA, 22 bp, synthetic construct
  source 1..22: mol_type = other DNA
gttggtacggtgccctgaaaga

SEQ ID NO: 10 - DNA, 22 bp, synthetic construct
  source 1..22: mol_type = other DNA
gctgacaggtaggacagacgat

SEQ ID NO: 11 - DNA, 22 bp, synthetic construct
  source 1..22: mol_type = other DNA
ggactgtggatgtcctgcgttt

SEQ ID NO: 12 - DNA, 22 bp, synthetic construct
  source 1..22: mol_type = other DNA
gccaattaccatcaaggctcgg

SEQ ID NO: 13 - DNA, 23 bp, synthetic construct
  source 1..23: mol_type = other DNA
ccttcaagacacagaggagaacc

SEQ ID NO: 14 - DNA, 22 bp, synthetic construct
  source 1..22: mol_type = other DNA
ctgtagtcgctggtgaatgtgc

SEQ ID NO: 15 - DNA, 1417 bp, Luoshenia tenuis
  source 1..1417: mol_type = genomic DNA
cttcggcgcctggctcccttgcgggttacctcaacgacttcgggcgctcccaactcccatggtgtgacgggcggtgtgtacaaggcccgggaacgcattcaccgcgacatgctgattcgcgattactagcaactccaacttcatgtgggcgggttgcagcccacaatccgaactgggaccggttttttgagatccgctccaccttacggtctcgcttctctttgtaccggccattgtagcacgtgtgtagcccaggtcataaggagcatgatgatttgacgtcgtccccaccttcctccgtgttatccacggcagtcctgttagagtccccggctttacccgctggtaactaactgtaggggttgcgctcgttgcgggacttaacccaacatctcacgacacgagctgacgacaaccatgcaccacctgtctcctcgtcccgaaggacgtacatatctctatgcacttcgagggatgtcaagacctggtaaggttcttcgcgttgcttcgaattaaaccacatgctccgctgcttgtgcgggcccccgtcaattcctttgagtttcaaccttgcggccgtactccccaggcggaatacttattgcgtttgctccggcacagggggggtcgataccccctacacctagtattcatcgtttacagcgtggactaccagggtatctaatcctgttcgctccccacgctttcgcgcctcagcgtcagttacagtccagaaagccgccttcgccactggtgttcctcctaatatctacgcatttcaccgctacactaggaattccgcttccctctcctgcactcctagccaaccagttttaaatgcagttcccaggttaagcccgggtatttcacatctaacttgatcggccgcctgcgcgccctttacgcccagtaattccggacaacgctcgccccctacgtattaccgcggctgctggcacgtagttagccggggcttcctcccatggtaccgtcacttccttcttcccataggacaaaagtttacaatccaaagaccttcttccttcacgcggcgttgctgggtcaggctttcgcccattgcccaatattccccactgctgcctcccgtaggagtctgggccgtgtctcagtcccagtgtggccggccaacctctcagtccggctacccatcgtcgccttggtaggcctttaccctaccaactagctaatgggacgcgagcccaccctgcaccggatttctcctttgacccctgtaccatgtgatactgtggtctcatgcggtattagcactgatttctcagtgttatccccctgtgcaggacaggttgctcacgcgttactcacccgtccgccactagatcagatgtctccatgccgaagcatttcctcatcctctcccgttcgactgcatggtaag

SEQ ID NO: 16 - DNA, 17 bp, synthetic construct
  source 1..17: mol_type = other DNA
cctacgggrsgcagcag

SEQ ID NO: 17 - DNA, 20 bp, synthetic construct
  source 1..20: mol_type = other DNA
ggactachvgggtwtctaat

(19) United States
(12) Patent Application Publication
(10) Pub. No.: US 2025/0177456 A1
(43) Pub. Date: Jun. 5, 2025

(54) METHOD AND COMPOSITION FOR PREVENTING AND/OR TREATING METABOLIC DISORDERS

(71) Applicant: SHANDONG UNIVERSITY, Qingdao (CN)

(72) Inventors: Shuangjiang LIU, Qingdao (CN); Chang LIU, Qingdao (CN); Yu JIANG, Qingdao (CN); Mengxuan DU, Qingdao (CN)

(21) Appl. No.: 18/526,931

(22) Filed: Dec. 1, 2023

(51) Int. Cl. A61K 35/741; A61K 35/00; A61P 1/16; A61P 3/04; A61P 3/10; C12N 1/20; C12R 1/01

(52) U.S. Cl. CPC A61K 35/741; A61P 1/16; A61P 3/04; A61P 3/10; C12N 1/205; A61K 2035/115; C12R 2001/01

(57) ABSTRACT

The present disclosure provides a method for preventing and/or treating metabolic disorders and a pharmaceutical composition. The method for preventing and/or treating metabolic disorders according to the present disclosure comprises administering an effective amount of Luoshenia tenuis to a subject. Further provided is a composition for preventing and/or treating metabolic disorders, comprising Luoshenia tenuis.

### SEQUENCE LISTING

The contents of the electronic sequence listing (6E28-2323208US-Sequence Listing.xml; Size: 17,056 bytes; and Date of Creation: Dec. 1, 2023) is herein incorporated by reference in its entirety.

### TECHNICAL FIELD

The present disclosure relates to the field of biomedical science, in particular to a method and a composition for preventing and/or treating metabolic disorders (including metabolic syndrome and obesity).

### BACKGROUND

During the past decades, the incidence of obesity and related metabolic disorders increased drastically and has become a worldwide major health concern imposing a heavy burden on the economy and public health. Anti-obesity medications such as glucagon-like peptide-1 (GLP-1), leptin, ghrelin and peptide tyrosine tyrosine (PYY) were designed based on gut-brain axis hormones and functioned by regulation of appetite and energy metabolism. However, the long-term pharmacotherapy of these chemical medications would cause adverse cardiovascular effects, increase suicidal risk and give rise to drug dependence. Alternatively, microecological therapy including probiotic, prebiotic and synbiotic has become a promising strategy for intervention of chronic diseases such as obesity, type 2 diabetes and constipation in respect of its low side effects. The microecological agent is basically comprised of viable microbes and the components and/or metabolites of those microbes which are prevalent and abundant in the normal gastrointestinal tracts of healthy people. As a result, the microecological therapy is well-tolerated for most of the patients and long-term application of which is rarely found to cause serious adverse effects. Thus, it is of practical value to develop more effective microecological therapies for chronic diseases such as obesity. We believe that as we have more in-depth knowledge about the compositional and functional differences of gut microbiomes between health and metabolic diseases, more microecological therapies will be developed.

Gut microbiome, as an ecosystem comprising large numbers of bacteria living in the intestinal tract, plays a pivotal role in maintaining host metabolism homeostasis, and its dysbiosis would cause or aggravate metabolic diseases as obesity, diabetes, atherosclerosis and fatty liver. Culture-independent gut metagenomic cohort studies concerning metabolic diseases have revealed a vast number of correlations between host pathologies and specific gut commensal microbial taxa, behind which might lie new biotherapies or pathogeneses for metabolic diseases. Functional study of these phenotype-associated taxa by culture-dependent studies will help us to understand host-microbe interactions and to develop new functional gut microbes into next-generation probiotics (NGPs) and clinical biotherapies. One successful example is Akkermansia muciniphila, one of the most promising NGPs, which has been demonstrated to counteract diet-induced obesity and related metabolic disorders such as insulin resistance, glucose intolerance and hepatic steatosis both using murine models [1, 2] and in clinic trial [3]. Another example is Christensenellaceae minuta, as its therapeutic potential on obesity and abnormal metabolism was experimentally validated in rodent models [4]. Some biotech companies turn to develop C. minuta into new biotherapy for management of obesity and associated metabolic disorders [5]. Besides, Faecalibacterium prausnitzii, Clostridium butyricum, Bacteroides xylanisolvens, Parabacteroides distasonis and Dysosmobacter welbionis were experimentally proven to contribute to host metabolism via known or unknown mechanisms [6-10]. However, due to the heavy workload and lack of culturable microbial resource, very limited number of commensal microbial taxa associated with host metabolic phenotypes have ever been functionally verified by wet-lab experiments with pure cultured strains to date [11]. There are still a large number of gut commensal microbes that are negatively correlated with metabolic diseases are functionally unknown. As increasing gut commensal bacteria associated with host health are cultured and studied, more functional candidates of NGPs will be unraveled.

### SUMMARY

In the present disclosure, we confirmed by in-silico analysis of gut metagenomic datasets that a novel gut commensal Luoshenia tenuis described in our previous study is widely existed in over 86% of the assessed healthy cohorts and significantly decreased in obese humans compared with the healthy counterparts [12]. We then verified that gavage of Luoshenia tenuis (L. tenuis) to high-fat diet (HFD) mice significantly decreased the body weight gain and food intake by modulation of genes involved in feeding behavior, and improved the glucose and lipid metabolism of diet induced obese (DIO) mice. The disclosure demonstrates that the novel human gut commensal L. tenuis is a promising candidate of NPGs for treatment of obesity and related metabolic disorders.

A first aspect of the present disclosure provides a method for preventing and/or treating metabolic disorders, comprising administering an effective dose of Luoshenia tenuis to a subject.

In some examples, the Luoshenia tenuis is active or inactivated.

In some examples, the Luoshenia tenuis is the NSJ-44 strain of Luoshenia tenuis deposited at China General Microbiological Culture Collection Center (CGMCC) under accession number CGMCC 40454.

In some examples, the Luoshenia tenuis is administered orally, intracolonically, intranasally, intrarectally, by catheter, by lavage, by nasogastric tube, by topical delivery, and/or by means of fecal microbiota transplantation (FMT).

In some examples, the metabolic disorders include one or more of body weight disorders, metabolic syndrome, disorders of glucose, or lipid metabolism disorders.

In some examples, the body weight disorders include one or more of overweight or obese.

In some examples, the metabolic disorders include one or more of hyperglycemia, type 2 diabetes, glucose intolerance, impaired glucose metabolism, and insulin resistance.

In some examples, the lipid metabolism disorders include one or more of hepatic steatosis or dyslipidemia.

In some examples, the subject is mammalian.

A second aspect of the present disclosure provides a composition, comprising Luoshenia tenuis.

In some examples, the Luoshenia tenuis is active or inactivated.

In some examples, the Luoshenia tenuis is the NSJ-44 strain of Luoshenia tenuis deposited at China General Microbiological Culture Collection Center (CGMCC) under accession number CGMCC 40454.

In some examples, the composition is a food composition or a pharmaceutical composition.

In some examples, the composition is a pharmaceutical composition, the pharmaceutical composition further comprises a pharmaceutically acceptable carrier.

In some examples, the pharmaceutical composition is formulated to be administered orally, intracolonically, intranasally, intrarectally, by catheter, by lavage, by nasogastric tube, by topical delivery, and/or by means of fecal microbiota transplantation (FMT).

In some examples, the pharmaceutical composition is used for preventing and/or treating metabolic disorders in a subject.

In some examples, the metabolic disorders include one or more of body weight disorders, metabolic syndrome, disorders of glucose, or lipid metabolism disorders.

In some examples, the food composition contributes to controlling body fat, contributes to maintaining a healthy blood lipid level, and/or contributes to maintaining a healthy blood glucose level.

A third aspect of the present disclosure provides an isolated NSJ-44 strain of Luoshenia tenuis deposited at China General Microbiological Culture Collection Center (CGMCC) under accession number CGMCC 40454.

### BRIEF DESCRIPTION OF THE DRAWINGS

FIG. 1. Biological and bioinformatical profiling of L. tenuis. (A) Electron microscopic pictures of L. tenuis; (B) The bar charts displaying the frequency of L. tenuis in the gut microbiomes of healthy adults (n=1,129); (C) The box-and-whiskers plot displaying the relative abundance (RA) of L. tenuis. in obese cohort (n=316) and healthy cohort (n=156); (D) The stacking histogram displaying the relative abundance of different classes of fermentation metabolites produced by in-vitro fermentation of L. tenuis in modified mGAM media by GC-MS; (E) The GC-MS scan image displaying the production of SCFAs by L. tenuis after 24-hour in-vitro fermentation in modified mGAM media. Peak 1, propionic acid; Peak 2, butyric acid; Peak 3, isobutyric acid; Peak 4, pentanoic acid.

FIG. 2 The intervention of L. tenuis alleviated body weight gain and food intake in high fat diet mice. (A) Experimental layouts and diet composition. (B) Body weight of mice treated during 17 weeks by daily oral gavage. (C) Average daily food intake at intervals of 3 days. (D) Inguinal fat weight of each group. (E) Transcription level of leptin, ghrl, and nucb2 in stomach. (F) Transcription level of pyy, nucb2 and geg in ileum. (G) Transcription level of pyy in colon. (H) The colonization test of L. tenuis in mice. *p<0.05: **p<0.01: ***p<0.001, ****p<0.0001.

FIG. 3 The changes of physiological and biochemical indexes in HFD mice after L. tenuis gavage. (A) and (B) Plasma glucose profile and mean AUC measured during an oral glucose tolerance test (OGTT). (C) and (D) Plasma glucose profile and mean AUC measured during insulin tolerance test (ITT). (E) and (F) Free and fasting blood glucose level at the end of gavage. (G) Appearance and sections of representative images from liver after H&E staining. Scale bars, 100 μm. Measurements were taken from distinct samples. (H) Liver weight and (I) Liver damage score of each group. (J) Appearance and sections of representative images of colorectum Swiss roll after H&E staining. (K) Colon length and (L) gut mucosa damage level of each group. The transcription level of (M) tjp1 and (N) cldn1. *p<0.05; **p<0.01.

FIG. 4. The impact of gut microbiota composition by L. tenuis gavage. (A) α diversity and (B) β diversity of each group in the cecal contents. (C) Microbial composition at phylum level of the cecal contents. (D) The log-transformed LDA scores computed with LEfSe for bacterial taxa differentially abundant between HFD_CK/LT groups.

FIG. 5. The intervention of L. tenuis improved glycolipid metabolism in DIO mice. (A) Experimental layouts and diet composition. (B) Periodic free diet blood glucose level. (C) Periodic fasting blood glucose level. (D) and (E) Plasma glucose profile and mean AUC measured during an OGTT. (F) and (G) Plasma glucose profile and mean AUC measured during ITT. (H) TG, (I) TCHO, (J) VLDL concentrations of plasma in groups. (K) ALT and (L) AST levels in plasma samples. (M) SOD activities in the liver. Concentration of (N) ZO-1, (O) TLR-4, (P) IL-6 and (Q) FGF-15 in colorectum. *p<0.05: **p<0.01: ***p<0.001, ****p<0.0001.

FIG. 6. The production of short chain fatty acids (SCFAs) by different gut microbes after 48-hour in-vitro fermentation in modified mGAM media. (A) The production of SCFAs by 3 gut microbes (μg/mL). (B) The GC-MS scan image displaying the production of SCFAs by 3 gut microbes. Peak 1, acetic acid: Peak 2, propionic acid: Peak 3, butyric acid: Peak 4, isobutyric acid: Peak 5, pentanoic acid.

### DETAILED DESCRIPTION

It must be noted that as used herein and in the appended claims, the singular forms “a”, “an”, and “the” include plural referents unless the context clearly dictates otherwise. Thus, for example, reference to “a peptide” includes a plurality of peptides.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

As used herein, the term “and/or” relates to and covers any and all possible combination of more than one item listed.

As used herein, the term “effective” means sufficient to realize a desired, anticipated, or required result as used in the description and/or the claims. When used in a case of treating a patient or object using a bacteria or composition, the term “effective amount”, “therapeutically effective amount”, “prevention effective amount”, or “pharmaceutically effective amount” refers to an amount of bacteria or composition which is, when administered to a subject or a patient to treat or prevent a disease, sufficient for such treatment or prevention of the disease.

The term “subject” used herein refers to a living mammalian organism, such as a human, a monkey, a cow, a sheep, a goat, a dog, a cat, a mouse, a rat, a guinea pig, or transgenic non-human species thereof. In some examples, the patient or subject is a primate. Some non-limiting examples of human patients are adults, adolescents, and infants.

“Preventing” or “prevention” includes: (1) suppressing onset of a disease in an object or patient that may be in the risk of the disease and/or is susceptible to the disease but has not yet undergone or exhibit any or all pathology or symptoms of the disease, and/or (2) slowing down onset of pathology or symptoms of a disease in an object or patient that may be in the risk of the disease and/or is susceptible to the disease but has not yet undergone or exhibit any or all pathology or symptoms of the disease.

“Treating” includes: (1) suppressing a disease in an object or patient that is currently undergoing or exhibiting pathology or symptoms of the disease (e.g., stopping further development of the pathology and/or symptoms), (2) alleviating a disease in an object or patient that is currently undergoing or exhibiting pathology or symptoms of the disease (e.g., reversing the pathology and/or symptoms), and/or (3) generating any measurable decrease of a disease or a symptom thereof in an object or patient that is currently undergoing or exhibiting pathology or symptom of the disease.

Herein, the term “improvement” refers to amelioration of a symptom related to a disease, and may refer to amelioration of at least one parameter for measuring or quantifying the symptom.

“Identity” refers to the sequence similarity between two polynucleotide sequences or two polypeptides. When positions of each of two sequences in comparison are occupied by the same basic group or amino acid monomer subunit, for example, if a position of two DNA molecules is occupied by adenine, the molecules are identical at the position. “Identity percentage” between two sequences refers to a function of a number of matching or identical positions shared by two sequences divided by a number of positions compared ×100. For example, upon optimal sequence alignment, if two sequences include 6 matching or identical positions in 10 positions, the two sequences will have 60% identical: if two sequences include 95 matching or identical positions in 100 positions, the two sequences will be 95% identical. Usually, two sequences compared are aligned to provide a maximum percentage of identity. For example, the comparison may be carried out by the BLAST algorithm, parameters of the algorithm selected to provide maximum matching between the sequences over the entire length of the reference sequences.

### Method for Treating Metabolic Disorders (Including Metabolic Syndrome and Obesity)

In some aspects of the present disclosure, provided is a method for preventing and/or treating metabolic disorders, comprising administering an effective amount of Luoshenia tenuis and/or a composition comprising Luoshenia tenuis to a subject.

<Luoshenia tenuis>

Herein L. tenuis refers to Luoshenia tenuis and is a novel gut commensal depicted in a prior research of the applicant [12] (Liu C, Du M X, Abuduaini R, et al. Enlightening the taxonomy darkness of human gut microbiomes with a cultured biobank. Microbiome. 9 (2021) 119., which is incorporated herein in its entirety). The isolated NSJ-44 strain that is a type strain of Luoshenia tenuis has been deposited under accession number CGMCC 1.32817 in China General Microbiological Culture Collection Center (CGMCC) on Apr. 2, 2021, address: NO.1 (3) West Beichen Road, Chaoyang District, Beijing 100101, China. Under the Budapest Treaty, the isolated NSJ-44 strain was deposited under accession number CGMCC 40454 in China General Microbiological Culture Collection Center (CGMCC) on Dec. 28, 2022, address: NO.1 (3) West Beichen Road, Chaoyang District, Beijing 100101, China. In some optional embodiments, the Luoshenia tenuis is not limited to the above deposited strains and may be a strain having substantively the same properties with the above deposited strains. The phrase “a strain having substantively the same properties” refers to a stain that is classified as L. tenuis and refers to a strain having at least the same degree of biological functions with the deposited stains of the present disclosure.

In some optional embodiments, the Luoshenia tenuis may include an isolated strain having a 16S rRNA sequence that is of at least 97% identify, preferably at least 98% identify, more preferably at least 98.5% identify, at least 98.7%, at least 99%, at least 99.5%, at least 99.9% identify to the 16S rRNA sequence of the isolated NSJ-44 strain. The 16S rRNA sequence of the isolated NSJ-44 strain is as below (NCBI registration No. NR_181383.1: SEQ ID NO: 15):

CTTCGGCGCCTGGCTCCCTTGCGGGTTACCTCAACGACTTCGGGCGCTCC
CAACTCCCATGGTGTGACGGGCGGTGTGTACAAGGCCCGGGAACGCATTC
ACCGCGACATGCTGATTCGCGATTACTAGCAACTCCAACTTCATGTGGGC
GGGTTGCAGCCCACAATCCGAACTGGGACCGGTTTTTTGAGATCCGCTCC
ACCTTACGGTCTCGCTTCTCTTTGTACCGGCCATTGTAGCACGTGTGTAG
CCCAGGTCATAAGGAGCATGATGATTTGACGTCGTCCCCACCTTCCTCCG
TGTTATCCACGGCAGTCCTGTTAGAGTCCCCGGCTTTACCCGCTGGTAAC
TAACTGTAGGGGTTGCGCTCGTTGCGGGACTTAACCCAACATCTCACGAC
ACGAGCTGACGACAACCATGCACCACCTGTCTCCTCGTCCCGAAGGACGT
ACATATCTCTATGCACTTCGAGGGATGTCAAGACCTGGTAAGGTTCTTCG
CGTTGCTTCGAATTAAACCACATGCTCCGCTGCTTGTGCGGGCCCCCGTC
AATTCCTTTGAGTTTCAACCTTGCGGCCGTACTCCCCAGGCGGAATACTT
ATTGCGTTTGCTCCGGCACAGGGGGGGTCGATACCCCCTACACCTAGTAT
TCATCGTTTACAGCGTGGACTACCAGGGTATCTAATCCTGTTCGCTCCCC
ACGCTTTCGCGCCTCAGCGTCAGTTACAGTCCAGAAAGCCGCCTTCGCCA
CTGGTGTTCCTCCTAATATCTACGCATTTCACCGCTACACTAGGAATTCC
GCTTCCCTCTCCTGCACTCCTAGCCAACCAGTTTTAAATGCAGTTCCCAG
GTTAAGCCCGGGTATTTCACATCTAACTTGATCGGCCGCCTGCGCGCCCT
TTACGCCCAGTAATTCCGGACAACGCTCGCCCCCTACGTATTACCGCGGC
TGCTGGCACGTAGTTAGCCGGGGCTTCCTCCCATGGTACCGTCACTTCCT
TCTTCCCATAGGACAAAAGTTTACAATCCAAAGACCTTCTTCCTTCACGC
GGCGTTGCTGGGTCAGGCTTTCGCCCATTGCCCAATATTCCCCACTGCTG
CCTCCCGTAGGAGTCTGGGCCGTGTCTCAGTCCCAGTGTGGCCGGCCAAC
CTCTCAGTCCGGCTACCCATCGTCGCCTTGGTAGGCCTTTACCCTACCAA
CTAGCTAATGGGACGCGAGCCCACCCTGCACCGGATTTCTCCTTTGACCC
CTGTACCATGTGATACTGTGGTCTCATGCGGTATTAGCACTGATTTCTCA
GTGTTATCCCCCTGTGCAGGACAGGTTGCTCACGCGTTACTCACCCGTCC
GCCACTAGATCAGATGTCTCCATGCCGAAGCATTTCCTCATCCTCTCCCG
TTCGACTGCATGGTAAG

In some preferable embodiments, the Luoshenia tenuis includes the isolated NSJ-44 strain. In some more preferably embodiments, the Luoshenia tenuis is the isolated NSJ-44 strain.

In some embodiments, the Luoshenia tenuis may be active Luoshenia tenuis or inactivated Luoshenia tenuis (e.g., heat-inactivated).

In some embodiments, the Luoshenia tenuis may be condensed or non-condensed, liquid, paste, semi-solid, or solid (e.g., pellet, granule, or powder), and may be frozen, dried, or freeze-dried (e.g., in a freeze-dried form or spray/fluid bed dried form).

### <Administration Pathway and Dosage>

In some embodiments of the present disclosure, the Luoshenia tenuis or a composition comprising Luoshenia tenuis may be administered orally, intracolonically, intranasally, intrarectally, by catheter, by lavage, by nasogastric tube, by topical delivery, and/or by means of fecal microbiota transplantation (FMT).

In some embodiments, the administration dosage and the number of times of administration of the Luoshenia tenuis or a composition comprising Luoshenia tenuis vary in accordance with the following factors: severity of the disease to be treated, administration pathway, and the age, the physical condition, and the reaction of the individual to be treated.

In some embodiments of the present disclosure, the Luoshenia tenuis or a composition comprising Luoshenia tenuis is administered at least once a week, preferably at least twice a week, more preferably at least three times a week. In some other embodiments, Luoshenia tenuis or a composition comprising Luoshenia tenuis is administered at least once a day or at least twice a day.

In some embodiments, Luoshenia tenuis or a composition comprising Luoshenia tenuis is administered in a period of 1 week, preferably in a period of 3 weeks, 5 weeks, 7 weeks, 9 weeks, 11 weeks, 13 weeks, 15 weeks, 17 weeks, 19 or more weeks.

In some embodiments, Luoshenia tenuis or a composition comprising Luoshenia tenuis is administered in a continuous period till a required result is achieved (e.g., the body weight is reduced, glucose or lipid metabolism disorders is alleviated (e.g., blood glucose or lipid is reduced).

In some embodiments, the administration of Luoshenia tenuis or a composition comprising Luoshenia tenuis is for life, i.e., not limited in terms of time.

In some embodiments of the present disclosure, a daily dosage of Luoshenia tenuis or a composition comprising Luoshenia tenuis administered on a daily basis is about 1×10² cfu/day to about 1×10¹⁵ cfu/day, a preferable daily dosage is about 1×10⁵ cfu/day to about 1×10¹² cfu/day, a more preferable daily dosage is about 1×10⁹ cfu/day, wherein cfu stands for “colony forming unit”.

### <Indications>

In some embodiments of the present disclosure, the term “metabolic disorders” refers to symptoms, diseases, and conditions caused by or characterized by: body weight disorders, abnormal weight gain, energy expenditure or consumption, change of reaction to intake or endogenous nutriment, energy, hormone, or other signaling molecules in the body, or change in metabolism of carbohydrates, lipids, proteins, nucleic acids, or a combination thereof. Metabolic disorders may be associated with lacking or excessive metabolic pathways causing metabolism disturbance of carbohydrates, lipids, proteins and/or nucleic acids. Examples of metabolic disorders include, but not limited to, metabolic syndrome, disorders of glucose, symptoms associated with insulin deficiency or insulin resistance, diabetes (e.g., type 2 diabetes), glucose intolerance, lipid metabolism disorders, atherosclerosis, hypertension, cardiac disease, stroke, nonalcoholic fatty liver disease, hyperglycemia, hepatic steatosis, dyslipidemia, immune system dysfunction associated with overweight and obesity, cardiovascular diseases, hyper cholesterol, increased triglyceride, asthma, sleep apnea, osteoarthritis, nerve degeneration, gallbladder diseases, X syndrome, inflammatory and immunologic diseases, dyslipidemia leading to atherosclerosis and cancer.

In some specific embodiments, the metabolic disorders include body weight disorders.

As used herein, the term “body weight disorders” refers to diseases associated with excessive body weight and/or enhanced appetite. Various parameters are used to determine whether or not a subject is overweight as compared with a reference healthy subject, the parameters including age, height, sex, and health condition of the subject. For example, the subject may be considered overweight or obese (or having obesity) based on the body mass index (BMI) of the subject. The body mass index is calculated by dividing the body weight (kg) of the subject with squared height (m) of the subject. An adult with BMI within the range of about 18.5 to about 24.9 kg/m² is considered to have a normal weight: an adult with BMI within the range of about 25 to about 29.9 kg/m² may be considered to be overweight (pre-obese); and adult with BMI of about 30 kg/m² or more may be considered as obese. Enhanced appetite often leads to excessive body weight. There are several conditions associated with enhanced appetite, such as the night eating syndrome featured by loss of appetite in the morning and overeating at night which may be associated with hypothalamus injury.

In some specific embodiments, the body weight disorders include overweight or obesity, such as weight gains induced by a high fat diet, overweight or obesity induced by a high fat diet, etc. As proven by Example 2, the method or composition provided by the present disclosure reduces weight gains and food intake of the subject. In the stomach, the method or composition provided by the present disclosure increases a transcriptional level of leptin and nucb2 respectively encoding LEPTIN and NESFATIN-1 and reduces a transcriptional level of ghrl encoding GHRELIN, thereby enhancing satiety, suppressing appetite, and reducing the stimulation for food intake. In the ileum, the method or composition provided by the present disclosure increases a transcriptional level of nucb2, pyy, and geg, thereby regulating appetite, suppressing gut hormone secretion by the pancreas, reducing food intake, and suppressing secretion of glucagon, etc.

In some specific embodiments, the metabolic disorders include metabolic syndrome. The term “metabolic syndrome” refers to a pathological condition of metabolic disorders for protein, fat, carbohydrate, and other substances in the human body, and is a complicated symptom complex of metabolic disorders. It is a dangerous factor leading to diabetes and cardiovascular diseases.

The metabolic syndrome is defined by at least three of the following five physical conditions:
Abdominal (central) obesity;
Elevated blood pressure;
Elevated fasting blood glucose;
High serum triglyceride;
Low high-density lipoprotein (HDL).

In some specific embodiments, the metabolic disorders include disorders of glucose.

The term “disorders of glucose” covers any condition featured by clinical symptoms or clinical symptom combination associated with increased glucose level and/or increased insulin level in a subject as compared with a healthy individual. Increased glucose level and/or increased insulin level may be manifested in the following diseases, symptoms, and conditions: hypoglycemia, type 2 diabetes, gestational diabetes, type 1 diabetes, insulin resistance, impaired glucose resistance, hyperinsulinemia, impaired glucose metabolism, pre-diabetes, other metabolic disorders (e.g., metabolic syndrome, also called X syndrome) and obesity.

In some specific embodiments, the disorders of glucose include one or more of hyperglycemia, type 2 diabetes, glucose intolerance, impaired glucose metabolism, and insulin resistance. As verified by Example 3, the method or composition provided by the present disclosure improves glucose metabolism in HFD mice. Further, as verified by Example 5, the administration of L. tenuis significantly improved hyperglycemia in DIO mice.

As used herein, the term “hyperglycemia” refers to the symptom of a subject of an increased amount of glucose circulating in the blood plasma as compared with a healthy subject. Hyperglycemia is diagnosable by a method known in the field, including measuring fasting blood glucose level as described herein.

As used herein, the term “glucose tolerance” refers to the ability of the subject to control blood glucose and/or serum insulin content when the intake of glucose fluctuates. For example, glucose tolerance includes the ability of the subject to reduce the blood glucose level to a level measured before glucose intake within about 120 minutes.

The terms “diabetes” and “diabetic” refer to the progressive disease associated with insufficient insulin production and typically featured by high blood glucose and glycosuria. The terms “pre-diabetes” and “pre-diabetic” refer to the condition that the subject does not have the typical characteristics or symptoms observed in diabetes, but have the characteristics or symptoms which may progress to diabetes without treatment.

Type 2 diabetes (T2D), formerly known as adult-onset diabetes, is a form of diabetes featured by high blood glucose, insulin resistance, and relative insulin deficiency. Type 2 diabetes may be diagnosed by determining an average blood glucose level by an A1C test, or may be diagnosed by a random glucose test, a fasting blood glucose test, or an oral glucose tolerance test. Type 2 diabetes is the most common form of diabetes and may be caused by various factors including obesity, lack of physical activities, and genetic causes.

As used herein, the term “insulin resistance” refers to the condition that a normal amount of insulin cannot produce normal physiological or molecular response. In some cases, endogenously produced or exogenous administered insulin exceeding a secretory volume can completely or partially overcome insulin resistance and produce biological repair.

In some specific embodiments, metabolic disorders include lipid metabolism disorders. In some specific embodiments, metabolic disorders include one or more of hepatic steatosis or dyslipidemia.

Hepatic steatosis, also called fatty liver disease, is a condition of the liver accumulating excessive fat. Hepatic steatosis may be nonalcoholic fatty liver disease (NAFLD) or alcoholic fatty liver disease. Nonalcoholic fatty liver disease is a common cause for chronic liver disease; and the global prevalence rate of NAFLD is constantly increasing along with the prevalence of obesity. Nonalcoholic fatty liver disease is the most common cause for elevated liver enzymes. Within the scope of NAFLD, usually only NAFLD progresses to cirrhosis and hepatocellular carcinoma. With the growing prevalence of obesity, the prevalence rate and impact of NAFLD continues to increase.

In some specific embodiments, hepatic steatosis also includes liver injury or inflammation caused by hepatic steatosis. As verified in Examples 3 and 5 of the present disclosure, the method or composition provided by the present disclosure ameliorates liver injury in HFD and DIO models.

In some embodiments, dyslipidemia is featured by abnormal lipid level in the blood, for example, increased plasmid cholesterol or triglyceride (TG) or both: elevated low-density lipoprotein (LDL) or very-low-density lipoprotein (VLDL) level: low high-density lipoprotein (HDL) level: or low HDL cholesterol level. In some embodiments, dyslipidemia refers to hyperlipidemia. Dyslipidemia may accelerate the progress of atherosclerosis.

As verified in Example 5 of the present disclosure, the method or composition provided by the present disclosure has anti-hyperlipidemic effects and reduces blood lipid in the DIO model.

In some embodiments of the present disclosure, the method or composition provided by the present disclosure ameliorates the mucosal barrier and reduces intestinal mucosa injury and inflammation in a subject having metabolic disorders.

### <Subject>

In some specific embodiments of the present disclosure, the subject is mammalian. In some optional embodiments, the subject is a human, a monkey, a cow, a sheep, a goat, a dog, a cat, a mouse, a rat, a guinea pig, or transgenic non-human species thereof. In some preferable embodiments, the subject is a human.

In some specific embodiments of the present disclosure, the subject is diagnosed with metabolic disorders such as body weight disorders, glucose metabolism disorders, and lipid metabolism disorders.

In some other specific embodiments of the present disclosure, the subject is in the risk of on-set of metabolic disorders (e.g., body weight disorders, metabolic syndrome, disorders of glucose, and lipid metabolism disorders). In some more specific embodiments, the risk corresponds to susceptibility such as genetic susceptibility to metabolic disorders (e.g., body weight disorders, metabolic syndrome, disorders of glucose, and lipid metabolism disorders).

### Composition

In some aspects of the present disclosure, provided is a composition comprising Luoshenia tenuis.

In some embodiments, the Luoshenia tenuis is active strains. In some other embodiments, the Luoshenia tenuis is inactivated strains.

In some embodiments, the type strain of Luoshenia tenuis is deposited at China General Microbiological Culture Collection Center (CGMCC) under accession number CGMCC 40454.

In some embodiments of the present disclosure the composition may be a food composition, such as a food additive that can be added to an edible material to prepare food products for human or animals. In some specific embodiments, examples of the food products include, but not limited to: fluid milk products such as milk and concentrated milk: fermented milk such as yogurt, sour milk, and frozen yogurt: milk powder: ice cream: cream cheeses: dry cheeses: soybean milk: fermented soybean milk: vegetable-fruit juices: fruit juices: sports drinks: confectionery: jelly: candies: health foods: animal feeds; and dietary supplements.

In some specific embodiments, the food composition may be a health food composition. In some specific embodiments, the food composition and/or the health food composition helps to control body fat, maintain a healthy level of blood lipid (cholesterol/triglyceride), and/or maintain a healthy level of blood glucose.

In some other embodiments of the present disclosure, the composition may be a pharmaceutical composition. In some specific embodiments, the pharmaceutical composition may be used for preventing and/or treating metabolic disorders.

In some embodiments, the pharmaceutical composition may be administered orally, intracolonically, intranasally, intrarectally, by catheter, by lavage, by nasogastric tube, by topical delivery, and/or by means of fecal microbiota transplantation (FMT).

In some specific embodiments, the pharmaceutical composition may be produced into a dosage form suitable for oral administration by a technology well-known by a person skilled in the art.

In some embodiments, the pharmaceutical composition may further comprise a pharmaceutically acceptable carrier widely used in the medicine manufacturing technology. For example, the pharmaceutically acceptable carrier may include one or more agent selected from solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. Selection and the number of these agents are within the competence and the routine technologies of a person skilled in the art.

In some embodiments, the pharmaceutical composition may be produced into a dosage form suitable for oral administration by a technology well-known by a person skilled in the art, the dosage form including, but not limited to: axenic powder, tablet, troche, lozenge, pellet, capsule, dispersible powder or granule, solution, suspension, emulsion, syrup, elixir, slurry, and the like.

In some embodiments, the administration dosage and the number of times of administration of the pharmaceutical composition vary in accordance with the following factors: severity of the disease to be treated, administration pathway, and the age, the physical condition, and the reaction of the individual to be treated. Generally speaking, the composition may be administered orally in a single dose or in multiple doses.

### Isolated Luoshenia Tenuis Strain

In some aspects of the present disclosure, provided is an isolated Luoshenia tenuis strain deposited at China General Microbiological Culture Collection Center (CGMCC) under accession number CGMCC 40454.

While the foregoing has described what are considered to be the best mode and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present teachings.

### Materials and Methods

### 1. Ethical Statements

All mice experiments in this study were approved by the ethics committee of Institute of Microbiology, Chinese Academy of Sciences (IMCAS). The protocols were approved by the Committee on the Ethics of Animal Experiments of IMCAS (permit SQIMCAS2021051). The experiments were conducted according to the National Institutes of Health Guidelines for the Care and Use of Laboratory Animals (NIH publications No. 8023, revised 1978).

### 2. Mice, Housing and Diets

All C57BL/6 J male mice were purchased from Beijing Vital River Laboratory Animal Technology Co., Ltd. (Beijing, China). The mice were housed in a specific-pathogen-free (SPF) environment under controlled conditions, a 12-h light/dark cycle, a temperature of 20-22° C., and 45±5% humidity, with free access to food and water. All mice were acclimated for one week before the experiment.

For the L. tenuis efficiency assay on HFD-feeding mice, five-week-old C57BL/6J male mice fed with HFD (D12492, 60% calories from fat, Research Diets, USA) for 16 weeks were sorted into three groups (n=15 each) based on their initial blood glucose levels and body weight. One group was treated daily with 1×10⁹ cfu of lived L. tenuis suspended in 0.1 mL of sterile anaerobic PBS by mouth. Another group was given the same amount of L. tenuis cells that were inactivated by autoclaving at 121° C. for 15 min. The control group was given an equivalent volume of sterile anaerobic PBS by gavage.

For the L. tenuis efficiency assay on DIO mice, five-week-old C57BL/6J male mice fed with HFD for 10 weeks to induce obesity by company (GemPharmatech LLC, Jiangsu, China), and the DIO mice with body weight >35 g were purchased and delivered to our P2 animal house with a controlled environment for an acclimation period of one-week. And then they were sorted into four groups based on their body weight and blood glucose levels and treated for another 9 weeks. The DIO_LT group (n=10) was treated daily with 1×10⁹ cfu of lived L. tenuis suspended in 0.2 mL of sterile anaerobic PBS by mouth. The DIO_LTpa group (n=5) was given the same amount of L. tenuis cells that were inactivated by autoclaving at 121° C. for 15 min. The control groups (n=5 each) DIO_CK and ND_CK were given an equivalent volume of sterile anaerobic PBS by gavage. But the diet of ND_CK group mice was changed from HFD to normal diet (ND).

### 3. Microbial Strains

Luoshenia tenuis was cultured in modified mGAM broth medium at 37° C. in an anaerobic tube for 48 h [12]. For the in vivo efficacy assay, cells were obtained by centrifuging at 8,000 g for 10 min at 4° C. The cells suspension for oral administration was prepared by suspending the cultured bacterial cells in sterile anaerobic PBS with a final cell density of 1×10⁹ CFU/mL. 100 μL of the bacterial suspension was given daily.

### 4. Body Weight and Food Intake

The body weight and food intake were measured every 3 days. Food intake per mouse per day was determined by the following equation: (total food intake each cage)/(5 mice per cage)/(days of food consumption).

### 5. Fecal Samples

Each mouse was kept in an empty compartmentalized storage boxes without bedding for 30 min, and then collected fresh fecal samples into sterile tubes. Tubes were stored at −80° C. for further analysis.

### 6. Tissue Sampling

After treatment, each mouse was anesthetized with pentobarbital sodium (TRC, Canada), and blood was sampled from the fundus portal and cava veins. After exsanguination, mice were euthanized by cervical dislocation. The intestines and the liver were precisely dissected, weighed and photographed. One piece of tissue (0.5 cm×0.5 cm square) in the middle of the largest lobe of liver were cut off and fixed in 4% paraformaldehyde for 24 h at room temperature and the other liver tissues were frozen in liquid nitrogen and stored at −80° C. for further analysis.

### 7. Insulin Tolerance Test (ITT) and Oral Glucose Tolerance Test (OGTT)

An ITT was performed by injecting insulin (0.6 U/kg) intraperitoneally after 6 h of fasting. An OGTT was performed by gavage of glucose solution (2 g/kg) after overnight fasting. The level of blood glucose was measured by tail vein bleeding using a glucose meter (EA-18, Sinocare, China) before oral glucose load (0 min) and at 30, 60, and 120 min after oral glucose load. The AUCs generated from the data collected during the ITT/OGTT were calculated with GraphPad v8.2.1.441.

### 8. Histological Analysis

Liver tissue was sampled promptly after euthanasia, and fixed in 4% paraformaldehyde until paraffin embedding. Colorectum was emptied and subsequently rolled around needle after which the generated Swizz rolls were carefully preserved in 4% paraformalin until paraffin embedding. The paraffin sections of liver, and colorectum tissues were stained with H&E. The slides were scanned under a light microscope using Imaging Sys (Nikon). Liver sections were prepared by company (Servicebio Co. Ltd., Hubei, China) and subsequently assessed by two independent observers in a blinded fashion, using the liver damage score for evaluation of H&E staining liver sections as described by previous study [13]. For colorectum intestinal injury score, morphologic examinations were performed using inverted fluorescence microscope, and colorectum injury was analysed by a blinded, and experienced investigator for absent, mild, moderate, or severe injury, according to Chiu's intestinal injury pathological grading (score 0-5) [14].

9. Real-Time qPCR Analysis

Total RNA was extracted and purified from stomach, ileum and colon tissues following the protocol described in the Blood and Tissue Kit and TRIzol reagent (Vazyme). Concentration of total RNA was tested by Qubit (Invitrogen, Q33226). The cDNA was synthesized using a HiScript III RT SuperMix reverse transcription kit (Vazyme). Gene expression levels were detected using SYBR Green I (Vazyme) and pre-designed primer (Table 1). The relative mRNA expression level normalized to the internal control β-actin gene was calculated by the comparative threshold cycle (Ct) method [15]. Experiments were performed in triplicate and repeated at least two times. The qPCR mixture contained 10 μL SYBR green DNA-binding dye, 1 μL cDNA of each tissue and 0.8 μL primers, then replenish with DEPC water to 20 μL PCR amplification was performed using the following cycling parameters: 3 min at 95° C., 40 cycles of 3 s at 95° C., and 30 s at 60° C.

TABLE 1
Primer sequences and annealing temperature
for qPCR

| Target | | |
|---|---|---|
| gene | | Primer sequence |
| leptin | Forward | GCAGTGCCTATCCAGAAAGTCC |
| | | (SEQ ID NO: 1) |
| | Reverse | GGAATGAAGTCCAAGCCAGTGAC |
| | | (SEQ ID NO: 2) |
| ghr1 | Forward | GAAGCCACCAGCTAAACTGCAG |
| | | (SEQ ID NO: 3) |
| | Reverse | CTGACAGCTTGATGCCAACATCG |
| | | (SEQ ID NO: 4) |
| nucb2 | Forward | AGACCGATTGGTGACTCTGGAG |
| | | (SEQ ID NO: 5) |
| | Reverse | CCTCGGTGAATAACTGTTGCTGG |
| | | (SEQ ID NO: 6) |
| pyy | Forward | GCCACTACCTCAACCTGGTCAC |
| | | (SEQ ID NO: 7) |
| | Reverse | TCGCTGTCGTCTGTGAAGAGCA |
| | | (SEQ ID NO: 8) |
| tjp1 | Forward | GTTGGTACGGTGCCCTGAAAGA |
| | | (SEQ ID NO: 9) |
| | Reverse | GCTGACAGGTAGGACAGACGAT |
| | | (SEQ ID NO: 10) |
| cldn1 | Forward | GGACTGTGGATGTCCTGCGTTT |
| | | (SEQ ID NO: 11) |
| | Reverse | GCCAATTACCATCAAGGCTCGG |
| | | (SEQ ID NO: 12) |
| gcg | Forward | CCTTCAAGACACAGAGGAGAACC |
| | | (SEQ ID NO: 13) |
| | Reverse | CTGTAGTCGCTGGTGAATGTGC |
| | | (SEQ ID NO: 14) |

### 10. In-Silico Analysis of Publicly-Accessible Metagenomic Datasets

The 1,129 gut metagenomes used for analysis of the prevalence and relative abundance of L. tenuis species in gut microbiomes of healthy humans were collected as described in our previous study in which the accessions of each run was available [12]. The publicly available metagenomic dataset form the cohort of obese patients and its healthy counterparts was available under Bioproject PRJEB4336, and was achieved directly from GMrepo [16]. The prevalence and relative abundance was analysed by kraken2-based annotation of quality-controlled raw metagenomic data using a customized database as described in our previous study [12].

11. 16S rRNA Gene Sequencing and Analysis

DNA for amplicon sequencing of gut microbiota was extracted from approximately 50 mg of cecum contents of mouse. The V3-V4 region of 16S rRNA was amplified using the primers F341 (CCTACGGGRSGCAGCAG; SEQ ID NO: 16) and R806 (GGACTACHVGGGTWTCTAAT; SEQ ID NO: 17) by PCR and sequenced in the HiSeq 2500 by company (Megagene, Guangzhou, China). The clean reads were denoised (command: -unoise3) and analyzed by Usearch 64-bit v11 following the recommended pipeline (https://drive5.com/usearch/). A representative sequence of each ASV (amplicon sequence variants) was assigned to the taxa at genus level in the ltp_vhGMB constructed in our previous work [12]. Statistical analysis of differentially abundant sequences and taxa were performed by MicrobiomeAnalyst (https://www.microbiomeanalyst.ca/MicrobiomeAnalyst/) [17].

### 12. Short-Chain Fatty Acid (SCFA) and Volatile Metabolites Analysis

For the measurement of SCFA, the levels of SCFAs (acetic acid, propionate, isobutyrate, butyrate, isopentanoic acid, and pentanoic acid) were measured by GC-MS according to a previously described method [12]. Briefly, the samples were extracted with equal volume of ethyl acetate by vortex for three times. All content transferred to 15 mL centrifuge tube, centrifuged for 10 min at 8000 rpm. Supernatants were collected in automatic injection bottle (2 mL 9 mm, ND9) for further analysis. The extracts were analysed using a QP 2010 Ultra GC-MS system with a Rtx-Wax capillary column (60 m×0.25 mm×0.25 mm), and the injection was performed at 230° C. with 2 mL of samples. Helium at a flow rate of 1.2 mL/minutes as used as the carrier gas. Electronic impact was recorded at 70 eV. Oven temperatures were programmed from 60° C. to 100° C. at 5° C./minute, with a 1-minute hold; to 150° C. at 5° C./minute, with a 5-minute hold; and to 225° C. at 30° C./minute, with a 20-minute hold.

For the measurement of volatile metabolites, the headspace-solid phase micro-extraction was performed as described by Chen et. al [18]. The samples were analysed using a QE 2104324S GC-MS system with an DB-5 MS capillary column (30 m×0.25 mm×0.25 μm), and the injection was performed at 240° C. Helium at a flow rate of 1 mL/minutes as used as the carrier gas. Electronic impact was recorded at 70 eV. Oven temperatures were programmed from 60° C. to 240° C. at 5° C./minute, with a 15-minute hold; and to 300° C. at 30° C./minute, with a 5-minute hold, and data were collected in full scan mode (m/z 32-500).

### 13. Statistical Analysis.

All data are expressed as the mean±SEM. Mouse groups were compared using one-way or two-way ANOVA, followed by either the Kruskal-Wallis or the LSD post-hoc test. Statistical analysis was performed using SPSS v20, and visualized using GraphPad Prism v8.2.1.441. Statistical significance is indicated by asterisks (*); *P<0.05, **P<0.01, ***P<0.001, ****P<0.0001.

### Example 1

### Biological and Bioinformatical Profiling of L. tenuis

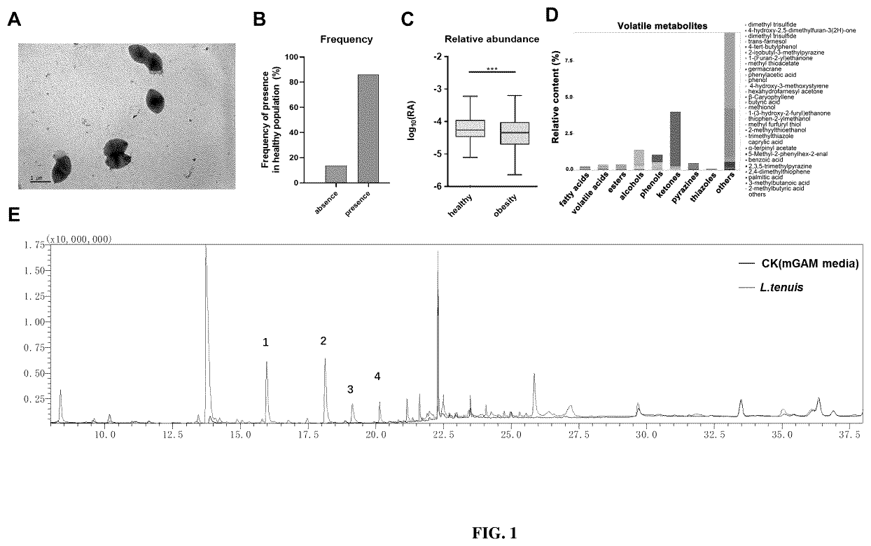

Luoshenia tenuis (NSJ-44), a strictly anaerobic microbe isolated from fecal sample of a healthy adult, was described and denominated in our recent study [12] The phylogenetic tree and genomic analysis revealed that Luoshenia was a new genus, closest to Christensenella minuta, and the 16S rRNA gene sequence similarity between L. tenuis and C. minuta was 87.07%. The bacterial colonies on modified mGAM agar plates were extremely small, showing pinpoint, whitish to semi-translucent, convex colonies. Image achieved by transmission electron microscope (FIG. 1A) displayed that the bacterial cells are ovoid with pointed ends (1.3-1.6 μm long and 0.7-0.9 μm wide), and occur singly or in pairs.

To investigate the prevalence of L. tenuis in the populations, 1,129 gut metagenomes of healthy cohorts with various ages, regions and dietary structures were collected from GMrepo for kraken2-based taxonomic analysis as described in Methods. The results revealed that the L. tenuis was widely existed in healthy humans as it was found in over 86% of the analysed samples (FIG. 1 B) with a mean relative abundance (RA) of 0.008%. To further evaluate the association between L. tenuis and host obese pathology, we revisited and analysed the gut metagenomes from an obese cohort (n=316) and its healthy counterparts (n=156). As shown in FIG. 1C, the L. tenuis was significantly depleted in obese cohort compared with the healthy counterparts. The observed negative correlation suggested a protective potential of L. tenuis against obesity.

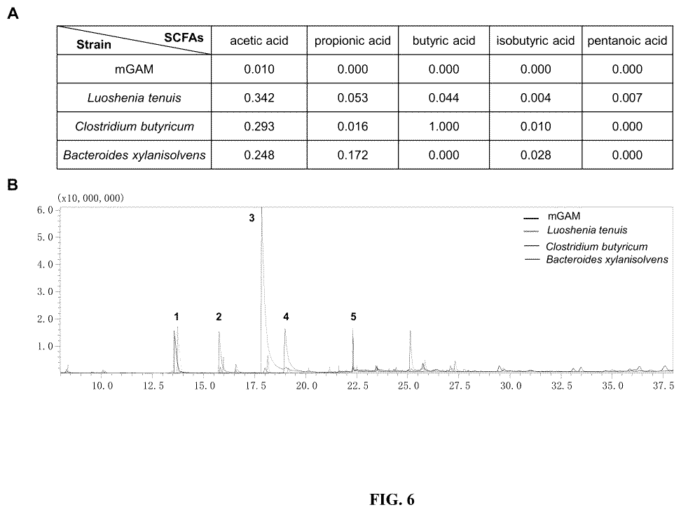

We then profiled the metabolic characteristics of L. tenuis by gas chromatography-mass spectrometry (GC-MS) analysis of the production of volatile metabolites including SCFAs during in-vitro fermentation. As shown in FIG. 1D, FIG. 6 and Table 2, L. tenuis produced trans-farnesol (v22) and hexahydrofarnesyl acetone (v28), which exerted anti-inflammatory effects as proved by previous studies [19-21]. Moreover, 2-acetylpyrazine (v30) and 4-Vinylguaiacol (v24) were reported to possess anti-tumor activity [22, 23]. We also verified that the L. tenuis was a producer of host-beneficial SCFAs, as propionic acid, butyric acid, isobutyric acid, pentanoic acid and isovaleric acid were identified after 24-hour fermentation of L. tenuis (FIG. 1E).

TABLe 2
The different classes of volatile metabolites produced by in-vitro fermentation
of L. tenuis in mGAM media by GC-MS

| | | | Base | | | | | | | |
|---|---|---|---|---|---|---|---|---|---|---|
| Num- | | RT/ | peak/ | | | | | | Relative | |
| ber | Type | min | m/z | Name | Formula | CAS# | Struction | Mw | content | Application |
| v1 | | 3.049 | 74.036 | Propionic acid | C5H₁₀O₂ | 105-37-3 | 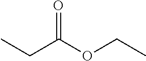 | 102.13 | 0.004 | Inhibit fungal growth |
| v2 | | 4.064 | 43.054 | Butyric acid | C₆H₁₂O₂ | 105-54-4 | 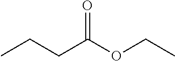 | 116.16 | 0.146 | Maintain a positive microecological balance in the gastrointestinal tract; provide a fast energy source |
| v3 | Fatty acids | 7.374 | 60.020 | Isovaleric acid | C₅H₁₀O₂ | 503-74-2 | 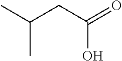 | 102.13 | 0.025 | Sedative; hypnotized |
| v4 | | 9.233 | 73.029 | Valeric acid | C₇H₁₄O₂ | 539-82-2 | 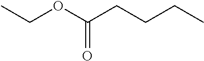 | 130.18 | 0.005 | Edible flavors; basic raw materials for organic synthesis |
| v5 | | 19.239 | 73.029 | Caprylic acid | C₁₀H₂₀O₂ | 106-32-1 | 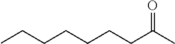 | 172.26 | 0.046 | |
| v6 | | 34.018 | 73.029 | Myristoleic acid | C₁₆H₃₂O₂ | 124-06-1 | 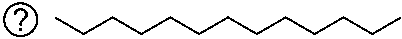 | 256.42 | 0.008 | Organic synthesis; edible flavor |
| v7 | | 38.024 | 73.029 | Palmitic acid | C₁₈H₃₆O₂ | 628-97-7 | 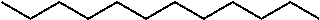 | 284.48 | 0.029 | |
| v8 | | 41.684 | 73.029 | Octa- decanoic acid | C₂₀H₄₀O₂ | 111-61-5 | 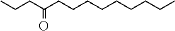 | 312.54 | 0.002 | Lubricants, emulsifiers; flavourings |
| v9 | Volatile acids | 7.235 | 102.067 | 2-Methyl- butyric acid | C₇H₁₄O₂ | 7452-79-1 | 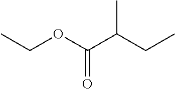 | 130.18 | 0.025 | Edible spices |
| v10 | | 10.042 | 61.011 | 2- Mercapto- propionic acid | C₅H₁₀O₂S | 19788- 49-9 | 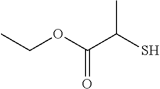 | 134.20 | 0.007 | Edible spices |
| v11 | | 18.417 | 105.034 | Benzoic acid | C₉H₁₀O₂ | 93-89-0 | 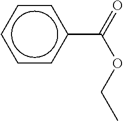 | 150.18 | 0.037 | Formulated fragrance; solvent and fragrance adjuvant; organic synthesis; preservatives |
| v12 | | 20.532 | 91.054 | Phenyl- acetic acid | C₁₀H₁₂O₂ | 101-97-3 | 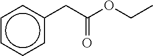 | 164.20 | 0.322 | Solvents and fragrance adjuvants, pharmaceutical intermediates |
| v13 | Esters | 5.097 | 43.017 | Methyl thioacetate | C₃H₆OS | 1534-08-3 | 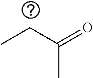 | 90.14 | 353 | Pharmaceutical, flavor and fragrance intermediates; acetyl thiol and thiol in organic synthesis |
| v14 | | 19.203 | 93.069 | Terpinyl acetate | C₁₂H₂₀O₂ | 80-26-2 | 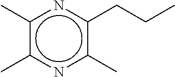 | 196.29 | 0.04 | Edible spices |
| v15 | Sulfides | 11.582 | 125.963 | Dimethyl trisulfide | C₂H₆S₃ | 3658- 80-8 | 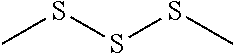 | 126.264 | 5.181 | Flavouring; cyanide antidote |
| v16 | | 19.733 | 157.935 | Dimethyl tetrasulfide | C₂H₆S₄ | 5756- 24-1 | 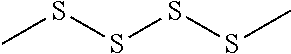 | 158.329 | 3.343 | Organic synthesis |
| v17 | | 7.046 | 61.011 | 2- Methyyl- thioethanol | C₃H₈OS | 5271- 38-5 | 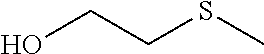 | 92.156 | 0.05 | Organic synthesis intermediates |
| v18 | | 11.016 | 95.050 | Methyl furfuryl thiol | C₆H₈OS | 59303- 05-8 | 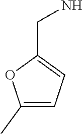 | 128.19 | 0.059 | Edible spices |
| v19 | Alcohols | 12.121 | 111.008 | 1-(3- hydroxy- 2- furyl) ethanone | C₆H₆O₃ | 3420-59-5 | 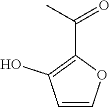 | 126.11 | 0.126 | Sugar alcohol sweetener |
| v20 | | 12.161 | 106.045 | 3- Methyl- thiopro- panol | C₄H₁₀ OS | 505-10-2 | 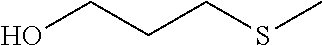 | 106.18 | 0.143 | Edible spices |
| v21 | | 13.917 | 85.011 | 2-Thio- pheneme thanol | C₅H₆OS | 636-72-6 | 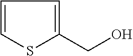 | 114.17 | 0.066 | Antithrombotic drug ticlopidine intermediate |
| v22 | | 32.361 | 41.038 | Trans- farnesol | C₁₅H₂₆O | 106-28-5 | 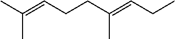 | 222.37 | 0.972 | Flavouring; antibacterial action |
| v23 | Phenols | 21.951 | 135.080 | 4-Tert- butylphenol | C₁₀H₁₄O | 98-54-4 | 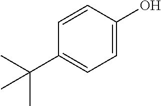 | 150.22 | 0.52 | Used to synthesize antioxidants, softeners, stabilizers |
| v24 | | 22.45 | 150.068 | 4-Hydroxy- 3- methoxy- styrene | C₉H₁₀O₂ | 7786- 61-0 | 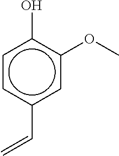 | 150.17 | 0.224 | Edible spice; germination inhibitor, with anti- inflammatory, anticancer activity. |
| v25 | | 12.266 | 94.0411 | Phenol | C₆H₆O | 108-95-2 | 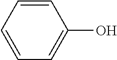 | 94.11 | 0.314 | Raw materials for the manufacture of resins, synthetic fibers, and plastics, as well as for the production of medicines and pesticides |
| v26 | | 11.074 | 102.014 | Tetra- hydro- thiophen- 3-one | C₄H₆OS | 1003- 04-9 | 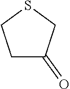 | 102.15 | 0.012 | Endogenous metabolites; Edible spices |
| v27 | Ketones | 15.184 | 128.047 | Furaneol | C₆H₈O₃ | 3658- 77-3 | 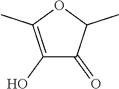 | 128.13 | 3.775 | Edible spices |
| v28 | | 35.02 | 95.086 | Hexahydro- farnesyl acetone | C₁₈H₃₆O | 502-69-2 | 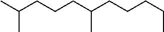 | 268.48 | 0.221 | Has antibacterial, analgesic and anti- inflammatory activity |
| v29 | | 35.244 | 105.034 | B-Phenyl propio- phenone | C₁₅H₁₄O | 1083- 30-3 | 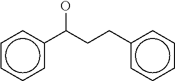 | 210.27 | 0.015 | Nutritious sweeteners; organic intermediates |
| v30 | Pyrazines | 13.612 | 94.053 | Acetyl- pyrazine | C₆H₆N₂O | 22047- 25-2 | 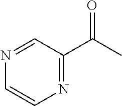 | 122.12 | 0.017 | Flavouring, a component of folic acid; antitumor |
| v31 | | 16.773 | 122.084 | 2,3,5- Trimethyl pyrazine | C₇H₁₀N₂ | 14667- 55-1 | 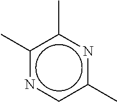 | 122.17 | 036 | Has antitumor, antibacterial and diuretic properties |
| v32 | | 17.33 | 108.068 | 2-Isobutyl- 3- methyl- pyrazine | C₉H₁₄N₂ | 13925- 06-9 | 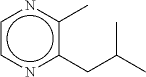 | 150.22 | 0.435 | Edible spices |
| v33 | | 8.991 | 112.022 | 2-Ethyl- thiazole | C₅H₇NS | 15679- 09-1 | 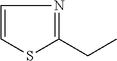 | 113.18 | 0.02 | Vulcanization accelerator |
| v34 | Thiazoles | 9.184 | 111.026 | 2,4- Dimethyl- thiophene | C₆H₈S | 638-00-6 | 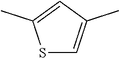 | 112.19 | 0.031 | Special odor |
| v35 | | 12.691 | 127.045 | Trimethyl thiazole | C₆H₉NS | 13623- 11-5 | 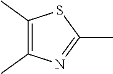 | 127.21 | 0.048 | Edible flavors; organic synthesis; pharmaceutical intermediates |
| v36 | Else | 26.211 | 41.038 | ß- Caryo- phyllene | C₁₅H₂₄ | 87- 44-5 | 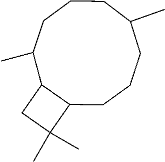 | 204.36 | 0.163 | CB2 receptor agonists; food flavor, anesthesia, treatment of colitis |
| v37 | | 33.093 | 69.070 | Germ- acrane | C₁₅H₃₀ | 645-10-3 | 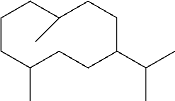 | 210.40 | 0.351 | Organic synthesis intermediates |
| v38 | | 9.471 | 95.013 | 2- Acetyl- furan | C₆H₆O₂ | 1192- 62-7 | 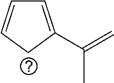 | 110.11 | 0.366 | Organic synthesis raw materials, pharmaceutical intermediates |
| v39 | | 26.981 | 115.054 | 5-Methyl-2- phenylhex- 2-enal | C₁₃H₁₆O | 21834- 92-4 | 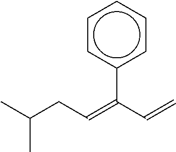 | 188.27 | 0.039 | Edible spices |

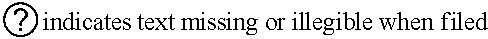

### Example 2

### L. tenuis Administration Reduces Body Weight Gain and Food Intake of HFD Mice

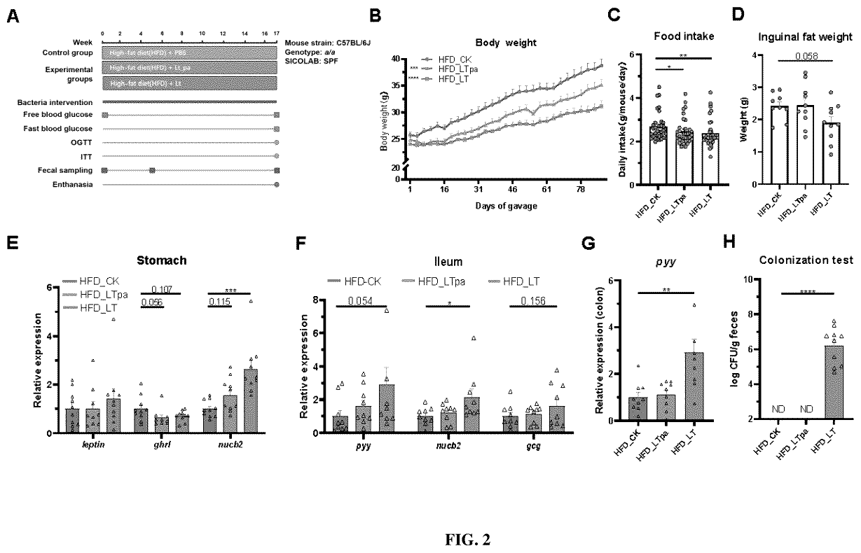

We then performed an animal trial as illustrated in FIG. 2A to experimentally verify the potential effects of L. tenuis on hosts. In brief, 5-week male C57BL/6J mice were daily administered with viable L. tenuis cells (HFD_LT, n=10), pasteurized L. tenuis (HFD_LTpa, n=10) and vehicle PBS (HFD_CK, n=10) for 17 weeks to monitoring the changes of body weight and the other metabolic features. First, compared with the control group, the body weight gain, daily food intake and inguinal fat weight in HFD mice were reduced significantly after L. tenuis administrations, which suggested a weight control effect of L. tenuis cells (FIGS. 2B-D). Besides, we noticed that the HFD mice treated with pasteurized L. tenuis also exhibited significant decrease on body weight gain and food intake, implying that the weight control effect was independent of cell viability.

To confirm if the reduced food intake was a microbe-mediated regulation of host behavior, we screened the marker genes/signalling pathways that were known to regulate or participate host food intake by qPCR-based quantification of gene transcriptions in gastrointestinal tissues of HFD mice. As shown in FIG. 2E, we found that the administration of viable and pasteurized L. tenuis increased the transcription level of gastric leptin and nucb2, respectively encoding the LEPTIN and NESFATIN-1 that were responsible for the increase of satiety and suppression of appetite in mice [24, 25]. Besides, we observed that the transcription level of ghrl encoding GHRELIN, a stimulant for feeding behavior, was decreased in HFD mice treated with viable and pasteurized L. tenuis compared with the control group (FIG. 2E). In ileum, the transcription level of nucb2 was increased significantly as in stomach. The transcription level of pyy and geg were increased clearly after L. tenuis intervention. The PYY encoded by pyy, a gut hormone that regulates appetite and inhibits pancreatic secretion. The GLP-1 encoded by geg, reducing food intake and inhibits glucagon secretion, which could further lead to the inhibition of pancreatic secretion and appetite (FIG. 2F). We also observed the transcription level of pyy was increased significantly in colon (FIG. 2G).

We next quantified the colonization of L. tenuis in HFD mice before and after gavage trial by qPCR analysis. Before gavage, the L. tenuis was not detected in the fecal samples of mice in each group. After 17-week gavage, the amount of L. tenuis was still undetectable in fecal samples of the HFD_CK and HFD_LTpa groups but significantly increased to 1.58±1.01*10⁶ cells/g dried faeces in HFD_LT group (FIG. 2H).

### Example 3

### L. tenuis Gavage Alleviates the Metabolic Disorders of HFD Mice

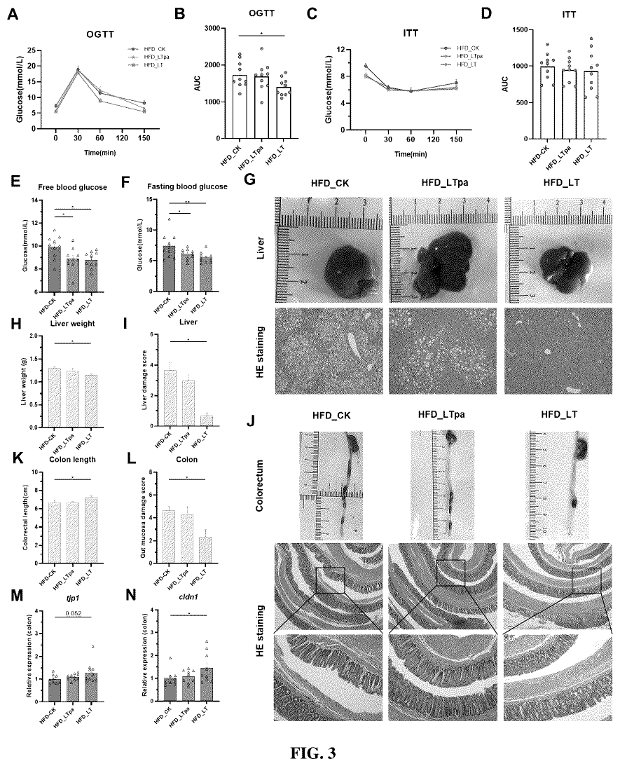

We then assessed the therapeutic effects of L. tenuis with respect to the pathoglycemia and liver damage of experimental mice induced by long-term HFD feeding [26]. As shown in FIGS. 3A and 3B, comparing with the HFD_CK group, the HFD_LT group showed significant hypoglycemic effect on oral glucose tolerance test (OGTT), and exhibited a marginal effect on the insulin tolerance test (ITT) (FIGS. 3C-D). And the free and fasted glycemia was decreased significantly in HFD_LT group comparing with the HFD_CK group (FIGS. 3E-F), showing that viable L. tenuis cells clearly improved blood glucose metabolism in HFD mice.

Further histological evaluation of liver tissue by the H&E staining revealed that the fatty-liver like hepatic lesions as macrosteatosis, hepatocyte ballooning and fat deposition was resorted after L. tenuis treatment comparing with the control group (FIG. 3G). The lower liver index and liver damage score in HFD_LT group also collaborated that the liver lesion was significantly improved by L. tenuis gavage (FIGS. 3H and 3I). Recent evidence supported that high fat diet affected intestinal mucosal barrier function [27]. Therefore, we investigated the effects of L. tenuis on the colorectum tissue. The results revealed that the gavage of living L. tenuis increased the average colorectum length (FIGS. 3J-K) and improved the gut barrier integrity as the mucosa damage score was decreased (FIG. 3L) and the transcription level of tjp1 and cldn1 encoding the tight junction protein zonula occludens 1 (ZO-1) and CLAUDIN-1 were increased (FIGS. 3M and 3N). The results above suggested that the administration of L. tenuis alleviated liver and intestinal mucosa damage in HFD mice.

### Example 4

### L. tenuis Administration does not Cause Apparent Disturbance on Gut Microbiota

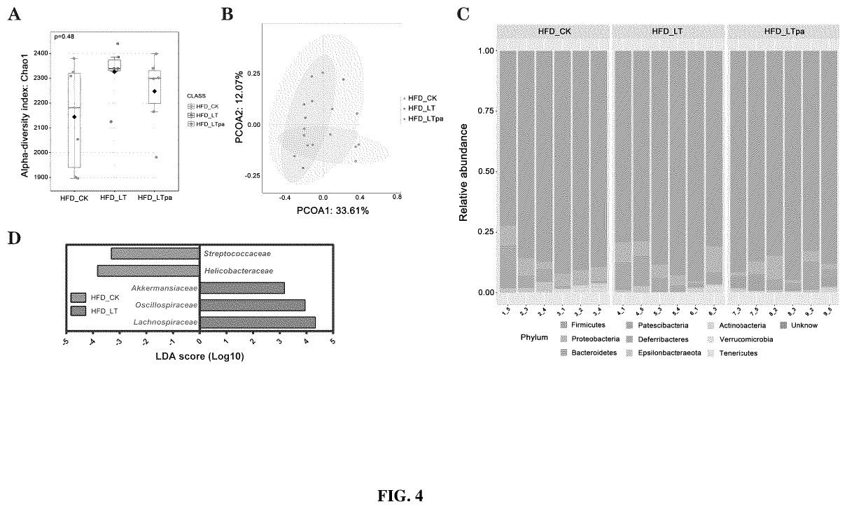

To evaluate if the gut microbiota composition was targeted and effected by L. tenuis gavage, we sequenced and analysed the 16S rRNA gene amplicons of caecal contents of experimental mice (n=6). As shown in FIG. 4A, the L. tenuis administration marginally increased the α diversity of gut microbiota as the Chao1 diversity index was increased after L. tenuis treatment. The subsequent principal coordinate analysis (PCoA) based analysis of β-diversity demonstrated that the gavage of viable and pasteurized L. tenuis slightly altered the overall gut microbiota composition (PERMANOVA or other statistical analysis, p<0.067) compared with the control group (FIG. 4B). Yet, the β-diversity between two intervention groups (HFD_LT and HFD_LTpa) showed no significant difference. There was no significant difference in the phylum-level microbial composition that was observed (FIG. 4C). Yet, further LDA Effect Size (LefSe) analysis demonstrated that the Lachnospiraceae, Oscillospiraceae and Akkermansiaceae were significantly enriched in HFD mice after L. tenuis treatment, while the Helicobacteraceae and Streptococcaceae were enriched in the control group (FIG. 4D). The results indicated that the impact of L. tenuis gavage on gut microbiota is very limited.

### Example 5

### L. tenuis Improves Glucose and Lipid Metabolism and Intestinal Barrier Function of DIO Mice

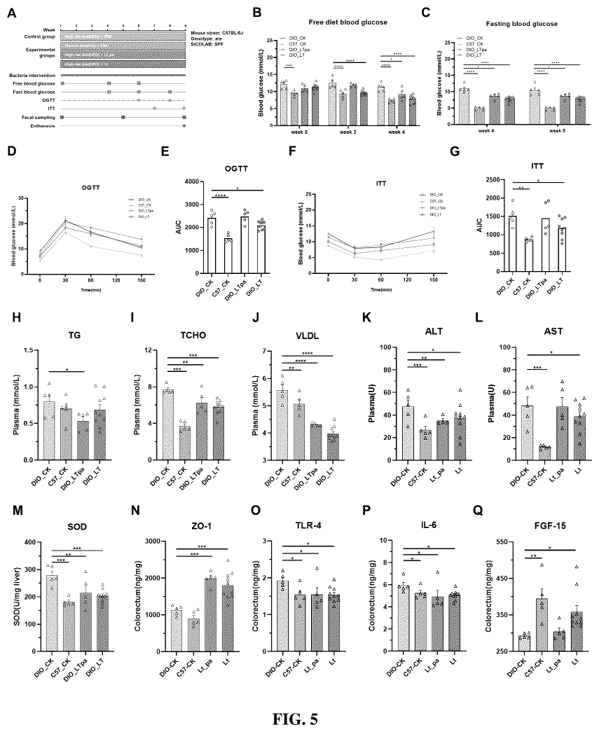

We have verified in last animal trial that the L. tenuis retarded the weight gain and metabolism deterioration in HFD mice. To evaluate the effects of L. tenuis on metabolic disorders, we used the DIO mice as animal models, and treated which by daily gavage of vehicle PBS (DIO_CK, n=5) and (C57_CK, n=5), viable L. tenuis (DIO_LT, n=10), and pasteurized L. tenuis (DIO_LTpa, n=5) for 9 weeks (FIG. 5A). The administration of L. tenuis significantly improved the hyperglycemia of DIO mice. The level of free and fasting blood glucose was significantly decreased after the administration of L. tenuis (FIGS. 5B and 5C). Moreover, the OGTT test revealed that the DIO mice treated with L. tenuis exerted a better glucose tolerance compared with the control group (FIGS. 5D and 5E). In parallel, the mice treated with viable L. tenuis exhibited a better insulin sensitivity than the DIO-CK mice did as demonstrated by the insulin tolerance test (ITT) test (FIGS. 5F and 5G).

Notably, the gavage of L. tenuis in DIO mouse model also exhibited antihyperlipidemic effect as the concentration of plasma triglyceride (TG), total cholesterol (TCHO), and very low density lipoprotein (VLDL) decreased significantly (FIGS. 5H-J). We also noticed that the administration of pasteurized L. tenuis also exerted hypoglycemic and hypolipidemic effects in DIO mice as the free and fasting blood glucose, TG, TCHO and VLDL were significantly reduced in HFD_LTpa group. Yet, its therapeutic efficacy was much weaker than that of viable L. tenuis. Moreover, we verified that the liver lesions of DIO mice were also alleviated after administration of either viable or pasteurized L. tenuis as the level of plasma alanine aminotransferase (ALT), plasma aspartate aminotransferase (AST) and hepatic superoxide dismutase (SOD) decreased in the DIO mice treated with viable or pasteurized L. tenuis (FIGS. 5K-M). The administration of L. tenuis exerted beneficial effects on the liver damage of DIO mice.

Moreover, we observed an increased level of intestinal epithelium tight junction protein ZO-1 and a decreased level of inflammatory factor toll-like receptor 4 (TLR-4) and interleukin-6 (IL-6) after administration of the viable or pasteurized L. tenuis compared with the control group (FIGS. 5N-P), suggesting that the gut epithelium barrier and inflammatory response of DIO mice was markedly improved after L. tenuis (viable or pasteurized) treatments. Finally, it was noteworthy that the administration of viable L. tenuis significantly upregulated the expression of fibroblast growth factor (FGF-15) (FIG. 5Q), a negative regulator of bile acid synthesis, gluconeogenesis and fatty acid oxidation in the liver, which might contribute to the observed improvement of glucose and lipid metabolism in DIO mice [28, 29].

Although the embodiments of the present disclosure have been described above, it will be appreciated that the above descriptions are merely exemplary, but not exhaustive; and that the disclosed embodiments are not limiting. A number of variations and modifications may occur to one skilled in the art without departing from the scopes and spirits of the described embodiments. The terms in the present disclosure are selected to provide the best explanation on the principles and practical applications of the embodiments and the technical improvements to the arts on market, or to make the embodiments described herein understandable to one skilled in the art.

### REFERENCE

1. Everard A, Belzer C, Geurts L, et al. Cross-talk between Akkermansia muciniphila and intestinal epithelium controls diet-induced obesity. Proc Natl Acad Sci USA. 110 (2013) 9066-9071. https://doi.org/10.1073/pnas. 1219451110.
2. Plovier H, Everard A, Druart C, et al. A purified membrane protein from Akkermansia muciniphila or the pasteurized bacterium improves metabolism in obese and diabetic mice. Nat Med. 23 (2017) 107-113. https://doi.org/10.1038/nm.4236.
3. Depommier C, Everard A, Druart C, et al. Supplementation with Akkermansia muciniphila in overweight and obese human volunteers: a proof-of-concept exploratory study. Nat Med. 25 (2019) 1096-1103. https://doi.org/10.1038/s41591-019-0495-2.
4. Goodrich J K, Waters J L, Poole A C, et al. Human genetics shape the gut microbiome. Cell. 159 (2014) 789-99. https://doi.org/10.1016/j.cell.2014.09.053.
5. Mazier W, Le Corf K, Martinez C, et al. A new strain of Christensenella minuta as a potential biotherapy for obesity and associated metabolic diseases. Cells-Basel. 10 (2021). https://doi.org/10.3390/cells10040823.
6. Le Roy T, Moens de Hase E, Van Hul M, et al. Dysosmobacter welbionis is a newly isolated human commensal bacterium preventing diet-induced obesity and metabolic disorders in mice. Gut. 71 (2022) 534-543. https://doi.org/10.1136/gutjnl-2020-323778.
7. Shilo S, Godneva A, Rachmiel M, et al. The gut microbiome of adults with type 1 diabetes and its association with the host glycemic control. Diabetes Care. 45 (2022) 555-563. https://doi.org/10.2337/dc21-1656.
8. Qiao S, Bao L, Wang K, et al. Activation of a specific gut bacteroides-folate-liver axis benefits for the alleviation of nonalcoholic hepatic steatosis. Cell Rep. 32 (2020) 108005. https://doi.org/10.1016/j.celrep.2020.108005.
9. Wang K, Liao M, Zhou N, et al. Parabacteroides distasonis alleviates obesity and metabolic dysfunctions via production of succinate and secondary bile acids. Cell Rep. 26 (2019) 222-235 e5. https://doi.org/10.1016/j.celrep.2018.12.028.
10. Sun J, Li H, Jin Y, et al. Probiotic Clostridium butyricum ameliorated motor deficits in a mouse model of Parkinson's disease via gut microbiota-GLP-1 pathway. Brain Behav Immun. 91 (2021) 703-715. https://doi.org/10.1016/j.bbi.2020.10.014.
11. Tanoue T, Morita S, Plichta D R, et al. A defined commensal consortium elicits CD8 T cells and anti-cancer immunity. Nature. 565 (2019) 600-625. https://doi.org/10.1038/s41586-019-0878-z.
12. Liu C, Du M X, Abuduaini R, et al. Enlightening the taxonomy darkness of human gut microbiomes a with cultured biobank. Microbiome. 9 (2021) 119. https://doi.org/10.1186/s40168-021-01064-3.
13. Kleiner D E, Brunt E M, Van Natta M, et al. Design and validation of a histological scoring system for nonalcoholic fatty liver disease. Hepatology. 41 (2005) 1313-1321. https://doi.org/10.1002/hep.20701.
14. Cui T, Miksa M, Wu R, et al. Milk fat globule epidermal growth factor 8 attenuates acute lung injury in mice after intestinal ischemia and reperfusion. Am J Respir Crit Care Med. 181 (2010) 238-246. https://doi.org/10.1164/rccm.200804-6250C.
15. Livak K J, Schmittgen T D. Analysis of relative gene expression data using real-time quantitative PCR and the 2(T)(−Delta Delta C) method. Methods. 25 (2001) 402-408. https://doi.org/10.1006/meth.2001.1262.
16. Dai D, Zhu J Y, Sun C Q, et al. GMrepo v2: a curated human gut microbiome database with special focus on disease markers and cross-dataset comparison. Nucleic Acids Res. 50 (2022) D777-D784. https://doi.org/10.1093/nar/gkab1019.
17. Chong J, Liu P, Zhou G Y, et al. Using MicrobiomeAnalyst for comprehensive statistical, functional, and meta-analysis of microbiome data. Nat Protoc. 15 (2020) 799-821. https://doi.org/10.1038/s41596-019-0264-1.
18. Chen J, Tang J N, Shi H, et al. Characteristics of volatile organic compounds produced from five pathogenic bacteria by headspace-solid phase micro-extraction/gas Microb. 57 (2017) 228-237. chromatography-mass spectrometry. J Basic https://doi.org/10.1002/jobm.201600505.
19. Shchepin R, Navarathna D H, Dumitru R, et al. Influence of heterocyclic and oxime-containing farnesol analogs on quorum sensing and pathogenicity in Candida albicans. Bioorg Med Chem. 16 (2008) 1842-1848. https://doi.org/10.1016/j.bmc.2007.11.011.
20. Wei G, Kong L, Zhang J, et al. Essential oil composition and antibacterial activity of Lindera nacusua (D. Don) Merr. Nat Prod Res. 30 (2016) 2704-2706. https://doi.org/10.1080/14786419.2015.1135145.
21. Avoseh O N, Mtunzi F M, Ogunwande I A, et al. Albizia lebbeck and Albizia zygia volatile oils exhibit anti-nociceptive and anti-inflammatory properties in pain models. J Ethnopharmacol. 268 (2021) 113676. https://doi.org/10.1016/j.jep.2020.113676.
22. Li M X, Chen C L, Ling C S, et al. Cytotoxicity and structure-activity relationships of four alpha-N-heterocyclic thiosemicarbazone derivatives crystal structure of 2-acetylpyrazine thiosemicarbazone. Bioorg Med Chem Lett. 19 (2009) 2704-2706. https://doi.org/10.1016/j.bmcl.2009.03.135.
23. Hunter W J, Manter D K, van der Lelie D. Biotransformation of ferulic acid to 4-vinylguaiacol by Enterobacter soli and E. aerogenes. Curr Microbiol. 65 (2012) 752-757. https://doi.org/10.1007/s00284-012-0222-4.
24. Schalla M A, Tache Y, Stengel A. Neuroendocrine peptides of the gut and their role in the Compr Physiol. 11 (2021) 1679-1730. regulation of food intake. https://doi.org/10.1002/cphy.c200007.
25. Dore R, Krotenko R, Reising J P, et al. Nesfatin-1 decreases the motivational and rewarding value of food. Neuropsychopharmacol. 45 (2020) 1645-1655. https://doi.org/10.1038/s41386-020-0682-3.
26. Lee Y S, Cabrales P, et al. Hepatocyte-specific HIF-1α ablation improves obesity-induced glucose intolerance by reducing first-pass GLP-1 degradation. Science Advances. 5 (2019) eaaw4176. https://doi.org/10.1126/sciadv.aaw4176.
27. Khuat L T, Le C T, Pai C S, et al. Obesity induces gut microbiota alterations and augments acute graft-versus-host disease after allogeneic stem cell transplantation. Sci Transl Med. 12 (2020). https://doi.org/10.1126/scitranslmed.aay7713.
28. Gadaleta R M, Moschetta A. Metabolic Messengers: fibroblast growth factor 15/19. Nat Metab. 1 (2019) 588-594. https://doi.org/10.1038/s42255-019-0074-3.
29. Xiang J W, Zhang Z Y, Xie H Y, et al. Effect of different bile acids on the intestine through enterohepatic circulation based on FXR. Gut Microbes. 13 (2021). https://doi.org/10.1080/19490976.2021.1949095.

What is claimed is:

1. A method for preventing and/or treating metabolic disorders, comprising administering an effective amount of Luoshenia tenuis to a subject.

2. The method according to claim 1, wherein the Luoshenia tenuis is active or inactivated.

3. The method according to claim 1, wherein the Luoshenia tenuis is a Luoshenia tenuis NSJ-44 strain deposited at China General Microbiological Culture Collection Center (CGMCC) under accession number CGMCC 40454.

4. The method according to claim 1, wherein the Luoshenia tenuis is administered orally, intracolonically, intranasally, intrarectally, by catheter, by lavage, by nasogastric tube, by topical delivery, and/or by means of fecal microbiota transplantation (FMT).

5. The method according to claim 1, wherein the metabolic disorders include one or more of body weight disorders, metabolic syndrome, disorders of glucose, or lipid metabolism disorders.

6. The method according to claim 5, wherein the body weight disorders include one or more of overweight or obesity.

7. The method according to claim 5, wherein the metabolic disorders include one or more of hyperglycemia, type 2 diabetes, glucose intolerance, impaired glucose metabolism, and insulin resistance.

8. The method according to claim 5, wherein the lipid metabolism disorders include one or more of hepatic steatosis or dyslipidemia.

9. The method according to claim 1, wherein the subject is mammalian.

10. A composition, comprising Luoshenia tenuis.

11. The composition according to claim 10, wherein the Luoshenia tenuis is active or inactivated.

12. The composition according to claim 10, wherein the Luoshenia tenuis is a Luoshenia tenuis NSJ-44 strain deposited at China General Microbiological Culture Collection Center (CGMCC) under accession number CGMCC 40454.

13. The composition according to claim 10, wherein the composition is a food composition or a pharmaceutical composition.

14. The composition according to claim 13, wherein the composition is a pharmaceutical composition, the pharmaceutical composition further comprises a pharmaceutically acceptable carrier.

15. The composition according to claim 14, wherein the pharmaceutical composition is formulated to be administered orally, intracolonically, intranasally, intrarectally, by catheter, by lavage, by nasogastric tube, by topical delivery, and/or by means of fecal microbiota transplantation (FMT).

16. The composition according to claim 13, wherein the pharmaceutical composition is for preventing and/or treating metabolic disorders in a subject.

17. The composition according to claim 16, wherein the metabolic disorders include one or more of body weight disorders, metabolic syndrome, disorders of glucose, or lipid metabolism disorders.

18. The composition according to claim 13, wherein the food composition helps to control body fat, maintain a healthy level of blood lipid, and/or maintain a healthy level of blood glucose.

19. A insolated Luoshenia tenuis NSJ-44 strain, which is deposited at China General Microbiological Culture Collection Center (CGMCC) under accession number CGMCC 40454.
